(19) United States
(12) Patent Application Publication
(10) Pub. No.: US 2023/0218645 A1
(43) Pub. Date: Jul. 13, 2023

(54) COMPOSITION CONTAINING CANNABINOIDS

(71) Applicant: HEALTHCANN SP. Z O.O., Wroclaw (PL)

(72) Inventors: Grzegorz Andrzej KIELBOWICZ, Le?ajsk (PL); Pawel MITULA, Wroclaw (PL); Tomasz TRONINA, Mi?dzybórz (PL)

(73) Assignee: HEALTHCANN SP. Z O.O., Wroclaw (PL)

(21) Appl. No.: 17/928,952

(22) PCT Filed: Jun. 1, 2021
(86) PCT No.: PCT/PL2021/050035

§ 371 Date: Dec. 1, 2022

(30) Foreign Application Priority Data
Jun. 1, 2020 (PL) P.434153

(51) Int. Cl. A61K 31/00; A61K 47/14; A61K 9/107; A61K 36/185; A61K 47/26; A23L 33/105; A23L 33/115

(52) U.S. Cl. CPC A61K 31/658; A61K 47/14; A61K 9/1075; A61K 36/185; A61K 47/26; A23L 33/105; A23L 33/115

(57) ABSTRACT

Self-emulsifying compositions are disclosed that form stable monodispersions in aqueous solutions containing relatively high concentrations of cannabinoids. They can be easily diluted in aqueous solutions. This makes them suitable for the production of water-based oral products, in particular pharmaceutical compositions, medical devices or food products.

An object of the invention is a self-emulsifying, stable composition containing cannabinoids, forming aqueous dispersion system of these organic compounds, which generally are known as insoluble in aqueous solutions. The proposed compositions can be easily diluted in aqueous solutions. This makes them suitable for preparation of oral products based on water, especially pharmaceutical compositions or food products.

Cannabinoids are natural compounds found in cannabis (Cannabis sativa L.)—both wild (Cannabis sativa ruderalis J.) or hemp (Cannabis sativa sativa L.) and indica (Cannabis sativa indica). Cannabis contains around 480 different substances, 80 of which are known as cannabinoids. The vast majority of them are devoid of psychoactive properties, while many of them show health-promoting properties. Currently, the greatest interest is in compounds such as tetrahydrocannabinol (THC), especially Δ⁹-THC and cannabidiol (CBD). Cannabinoids and cannabinoid extracts are insoluble in water. When placed in water or aqueous products in an unmodified form, they precipitate in the form of crystals falling to the bottom of the product or form a heterogeneous mixture, which leads to uneven distribution throughout the entire volume of the aqueous solution. As such, their oral bioavailability is low (usually less than 6%). Moreover, the delivered doses of cannabinoids are not the same, which is important for food products, where it is crucial that the effect is experienced as soon as possible after consumption. This negatively affects the homogenity of the product, its visual aspects and even dosage of cannabinoids when taken orally. Beyond beverages, many food products are water-based or require the presence of water in their production. Therefore, in order to be able to use cannabinoids in this type of products and obtain a uniform dosage, they must be dissolved. The use of cannabinoids in liquid products—aqueous solutions—requires their chemical modification or the development of an appropriate composition, which aims to create a stable suspension after their addition to aqueous systems.

Cannabinoid compositions that improve the solubility of cannabinoids in aqueous solutions are known in the state of art. They are used in the pharmaceutical industry, as well as in the food industry as additives to food products.

Formulations related to a self-dispersing emulsion using cannabinoids as a hydrophobic compound are known from patent specification US20200037638. The compositions disclosed increase the solubility of cannabinoids in aqueous solutions administered orally. The composition disclosed in the patent application contains a mixture of medium chain triglycerides and/or long chain triglycerides, a surfactant and cannabinoids. Known formulations are not without disadvantages: their dispersion time exceeds 60 s, and the obtained dispersions are not clear enough (transmittance below 85%), they are often characterized by high heterogeneity or too high polydispersity.

The main technical problem is the poor solubility of cannabinoids in aqueous solutions. Another problem of the currently used emulsifying systems, despite formation of a homogeneous suspension, is the change in visual properties of the products, i.e. the formation of the so-called “milky” suspensions unacceptable in many food products such as mineral waters, colourless and clear drinks. The cannabinoid insolubility leads to delamination and thus low stability of the emulsion over time. In order to prepare a dispersed system, extensive mixing and the use of devices such as sonicators or homogenizers are often necessary. One of the major problems is also to obtain an aqueous cannabinoid solution, which would be characterized by a small particle size and monodispersity in the aqueous system. An additional issue is the reduced speed of action of cannabinoids after oral ingestion, which is directly related to delamination of the emulsion.

Another problem is to provide an easily obtainable, stable aqueous cannabinoid suspension that could be used as a concentrate suitable for preparation of beverages being aqueous solutions that contain a well-defined, ultimately significantly diluted, content of cannabinoids. The known disadvantages of the current solutions are: the lack of resistance of the concentrated cannabinoid system to dilution, the lack of stability of products subjected to the preservation such as pasteurization, or the lack of stability at low pH (below 3), which significantly hinder production and processing of such concentrates.

The object of the present invention is to provide an improved composition, through which a stable nanoemulsion of water-insoluble cannabinoids is obtained, which is also characterized by high transparency, short time of forming a homogeneous dispersion system of cannabinoids, no need for extensive mixing to prepare a dispersion system, obtaining nanoparticles with a small hydrodynamic diameter, monodispersity of the nano-system, high dilution resistance in aqueous solutions, a wide pH range from pH=8 to pH<3, and final product preservation processes (e.g. pasteurization, cold filtering, UV radiation).

For the purposes of the present application, the high transparency of nanoemulsion when added to an aqueous solution means a transparency of greater than 85%, preferably greater than 90%. In the context of the invention, transparency should be equated with optical transmittance. The transmittance of the nanoemulsion obtained according to the invention can be measured as a percentage (% T) at a wavelength of λ=600 nm with any spectrometer in this range. In the studies described, measurements were made using a Merck® spectrometer model Pharo 300.

For the purposes of the present application, the short time to obtain a homogeneous dispersion system of cannabinoids means less than 60 seconds from the moment of adding the last drop of the mixture of the composition ingredients to water until complete dissolution. Measurement of the dispersion time can be performed by spectrometry using any spectrometer in the range 550-650 nm, wherein the dispersion time means achieving a constant value of transmittance or absorbance that does not change when the dispersion system is stirred for a long time under constant mixing conditions. Measurement of the dispersion time can also be performed using automatic analyzers of the stability and aging of emulsions, dispersions and suspensions, e.g. Turbiscan devices.

For the purposes of the present application, no need for extensive mixing to form a dispersion system means no need for using high shear mixing above 2500 RPM, homogenizers, high pressure homogenization and sonication methods. The application shows that mixing below 150 RPM provides adequate product parameters for the claimed compositions. Higher mixing value (range tested up to 2500 RPM) shortens the time and improves the quality of dispersion systems formed for the claimed cannabinoid-containing compositions. The mixing speed in this case is a relative concept, since the quality of mixing is influenced, among others, by the type and design of mechanical agitator (e.g. paddle, cup, anchor, frame, spiral, propeller, turbine, ribbon and other specialized agitators such as Visco-Jet, or even high-speed mechanical agitators), the construction of the mixer, reactor (e.g. the use of scrapers, baffles, wave breakers) or tank mixers.

For the purposes of the present application, the nanoparticles with small hydrodynamic diameter mean nanoparticles with a hydrodynamic diameter less than 180 nm. The value of the hydrodynamic diameter is defined in nanometers (D_{H}, nm), and its measurement can be performed with analyzers that determine particle size distribution using dynamic light scattering, e.g. Zetasizer Nano-ZS from Malvern.

For the purposes of the present application, the monodispersity of a nanosystem means the value of particle polydispersity (known as PdI—Polidispersity Index) after dispersion in water is less than 0.250. Measurement of the particle polydispersity after dispersion in water expressed as PdI value can be performed with Zetasizer Nano-ZS from Malvern.

For the purposes of the present application, the high dilution resistance in aqueous solutions should be equated with high stability of nanoemulsions at very high dilutions, i.e. dilutions up to 10,000×. Measurement of the nanoemulsion stability at 10,000× dilution can be performed by spectrometry with any spectrometer in the range 550-650 nm, wherein storage stability is characterized by a constant value of transmittance and/or absorbance. The emulsion stability can also be tested using Turbiscan analysers of emulsion stability and aging. Moreover, a constant value of the hydrodynamic diameter and the nanoparticle polydispersity in the system proves that the emulsion is stable and no aggregation, sedimentation or flocculation processes take place. Estimation of the hydrodynamic diameter and the nanoparticle polydispersity can be performed using analyzers that measure particle size distribution using dynamic light scattering, e.g. Zetasizer Nano-ZS from Malvern.

Unexpectedly, the above-defined complex technical goal has been achieved with the present invention.

The subject of the invention is a self-emulsifying, stable composition containing a cannabinoid or cannabinoid extract, a surfactant and two different lipid fractions. Preferably, the composition according to the invention consists of at least one cannabinoid or cannabinoid extract, a surfactant and two different lipid fractions.

In a preferred embodiment, the composition is characterized in that the cannabinoid or the cannabinoid extract constitutes no more than 20% by weight of the composition, the surfactant constitutes 30-50% by weight of the composition, the first lipophilic fraction constitutes 20-35% by weight of the composition, the second lipophilic fraction constitutes 15-30% by weight of the composition.

In a preferred embodiment, the composition is characterized in that the at least one cannabinoid or cannabinoid extract comprises tetrahydrocannabinol (Δ⁹-THC), cannabidiol (CBD), extract of Cannabis sativa, Cannabis indica, Cannabis hybrid and other Cannabis species, tetrahydrocannabinolic acid (THC-A), cannabidiolic acid (CBDA), cannabinol (CBN), cannabigerol (CBG), cannabigerolic acid (CBG-A), cannabichromene (CBC), tetrahydrocannabivarin (THC-V), Δ⁸-tetrahydrocannabinol (Δ⁸-THC), cannabidivarin (CBDV), cannabicyclol (CBL).

In a preferred embodiment, the composition is characterized in that the surfactant is polysorbate 80, the first lipophilic fraction is a medium chain length C8-C12 triacylglyceride, the second lipophilic fraction is a medium chain length C8-C12 mono-diacylglyceride.

In a preferred embodiment, the composition is characterized in that it forms a self-emulsifying clear, monodisperse system in an aqueous solution.

In a preferred embodiment, the composition is characterized in that the monodisperse system forms particles smaller than 180 nm.

In a preferred embodiment, the composition is characterized in that the monodisperse system is formed in less than 60 seconds and does not require extensive mixing.

In a preferred embodiment, the composition is characterized in that the monodisperse system forms a dilution-resistant stable nanoemulsion.

In a preferred embodiment, the composition is characterized in that it comprises flavour-enhancing additives and dyes.

A further subject of the invention is the use of the composition of the invention as defined above for preparation of water-based oral products. Preferably, the oral product is a pharmaceutical composition or a food product.

Another subject of the invention is a method for production of a stable monodisperse system, characterized in that water or an aqueous solution is mixed with the composition of the invention as defined above.

Preferably, the mixing time is less than 120 seconds, preferably less than 60 seconds, with mixing intensity not exceeding 2500 RPM, preferably with mixing intensity not exceeding 150 RPM.

Another subject of the invention is a monodisperse system characterized in that it consists of a dispersion medium being water or an aqueous solution and a dispersed phase formed by the particles obtained from the composition of the invention as defined above, wherein the particles of the invention having an outer layer containing a surfactant, and an inner layer which is a mixture of lipids in which a cannabinoid or cannabinoid extract is dissolved.

In the context of the present invention, the surfactants are surface-active chemical compounds, having the ability to adsorb on the surface of a system (phase boundary), that have both hydrophobic and hydrophilic properties in their molecule, associated with its amphiphilic structure: a part of the molecule has moieties of low affinity to the solvent, while another part—moieties of strong affinity to the solvent. Due to this structure, surfactants that have the ability to lower the surface tension even at a low concentration in the solution, as well as they have the ability to form micelles.

The surfactant component of the formulation can be used either alone or in combination with another surfactant to improve the self-emulsifying properties of the formulation. Preferred surfactant components are selected from the group consisting of:
polyglycolized glycerides and polyoxyethylene glycerides of medium- to long-chain mono-, di-, and triglycerides, such as: almond oil PEG-6 esters, almond oil PEG-60 esters, apricot kernel oil PEG-6 esters (Labrafil® M 1944 CS), caprylic/capric triglycerides PEG-4 esters (Labrafac® Hydro WL 1219), caprylic/capric triglycerides PEG-4 complex (Labrafac® Hydrophile), caprylic/capric glycerides PEG-6 esters (Softigen® 767), caprylic/capric glycerides PEG-8 esters (Labrasol®), castor oil PEG-35 esters (Etocas 35), hydrogenated castor oil PEG-40 esters (Croduret™ 40), hydrogenated castor oil PEG-50 esters (Croduret™ 50), hydrogenated castor oil PEG-5 esters, hydrogenated castor oil PEG-7 esters, ydrogenated castor oil PEG-9 esters, corn oil PEG-6 esters (Labrafil® M 2125 CS), corn oil PEG-8 esters (Labrafil® WL 2609 BS), corn glycerides PEG-60 esters, olive oil PEG-6 esters (Labrafil® M 1980 CS), hydrogenated palm/palm kernel oil PEG-6 esters (Labrafil® M 2130 CS), palm kernel oil PEG-40 esters, peanut oil PEG-6 esters (Labrafil® M 1969 CS), glyceryl laurate/PEG-32 laurate (Gelucire® 44/14), glyceryl laurate/PEG-20 laurate, glyceryl laurate/PEG-40 laurate, glyceryl oleate/PEG-20 oleate, glyceryl oleate/PEG-30 oleate, glyceryl palmitostearate/PEG-32 palmitostearate (Gelucire® 50/13), glyceryl stearate/PEG-32 stearate (Gelucire® 53/10), saturated polyglycolized glycerides (Gelucire® 37/02 and Gelucire 50/02), triisostearin PEG-6 esters (Labrafil® Isostearique), triolein PEG-6 esters, trioleate PEG-25 esters, polyoxyl 35 castor oil (Cremophor® EL), polyoxyl 40 hydrogenated castor oil (Cremophor® RH 40), polyoxyl 60 hydrogenated castor oil (Cremophor® RH60), and mixtures thereof;
polyglycolized derivatives and polyoxyethylene derivatives of medium- to long-chain fatty acids: PEG-8 caproate, PEG-8 caprylate, PEG-8 caprate, PEG-8 laurate, PEG-8 oleate, PEG-8 stearate, PEG-9 caproate, PEG-9 caprylate, PEG-9 caprate, PEG-9 laurate, PEG-9 oleate, PEG-9 stearate, PEG-10 caproate, PEG-10 caprylate, PEG-10 caprate, PEG-10 laurate, PEG-10 oleate, PEG-10 stearate, PEG-10 laurate, PEG-12 oleate, PEG-15 oleate, PEG-20 laurate, PEG-20 oleate, and mixtures thereof;
acyl polyglyceryl derivatives: polyglyceryl oleate, polyglyceryl-2 dioleate, polyglyceryl-3 dioleate (Plurol® Oleique from GATTEFOSSE), polyglyceryl-10 trioleate, polyglyceryl-10 laurate, polyglyceryl-10 oleate;
polyethylene glycol sorbitan fatty acid esters: PEG-20 sorbitan monolaurate (Tween 20), PEG-20 sorbitan monopalmitate (Tween 40), PEG-20 sorbitan monostearate (Tween 60), PEG-20 sorbitan monooleate (Tween 80), PEG-20 sorbitan tristearate (Tween 65), PEG-20 sorbitan trioleate (Tween 85) and mixtures thereof.
polyoxyethylene-polyoxypropylene block copolymers: poloxamers (108, 124, 182, 183, 188, 212, 217, 238, 288, 331, 338, 335, and 407), and mixtures thereof.
sorbitan fatty acid esters: sorbitan monolaurate, sorbitan monopalmitate, sorbitan monoleate, sorbitan monostearate and sorbitan tristearate, and mixtures thereof.

Other preferred surfactants, which can be used include TPGS (d-a-tocopheryl polyethylene glycol 1000 succinate), polyethyleneglycol 660 12-hydroxystearate (Solutol® HS-15), and mixtures thereof.

In the context of the present invention, the “first lipid fraction” are triacylglycerols of medium-chain (C8÷C12) and long-chain (C>12) saturated and unsaturated fatty acids, including hydrogenated fatty acids, and esters of fatty acids other than glycerols, and free fatty acids and derivatives thereof.

To improve the solubility of the lipophilic drug, the lipid fraction of the formulation can be selected from the group consisting of one or more of long-chain triglycerides or medium-chain triglycerides such as: anise oil, apricot kernel oil, beeswax, borage oil, canola oil, castor oil, cinnamon oil, clove oil, coconut oil, coconut oil-lecithin, coconut oil fractioned, coriander oil, corn oil, cottonseed oil, cottonseed oil hydrogenated, kernel oil, lemon oil, mineral oil, mineral oil (light), neutral oil, nutmeg oil, olive oil, orange oil, palm kernel oil, palm kernel oil hydrogenated, peanut oil, rapeseed oil, peppermint oil, poppy seed oil, safflower oil, sunflower oil, soybean oil, linseed oil, hemp oil, avocado oil, soybean oil hydrogenated, soybean oil refined, triolein, trilinolein, trilinolenin, and mixtures thereof.

Long-chain saturated fatty acids can be selected from the group consisting of: arachidic acid, behenic acid, 3-hydroxymyristic acid, lauric acid, lignoceric acid, mycoceranic acid, myristic acid, palmitic acid, phytanic acid, stearic acid and mixtures thereof.

Long-chain unsaturated fatty acids can be selected from the group consisting of: crotonic acid, myristoleic, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, dihomo-γ-linolenic acid, eicosatrienoic acid, stearidonic acid, arachidonic acid, eicosatetraenoic acid, adrenic acid, bosseopentaenoic acid, eicosapentaenoic acid, ozubondo acid, sardine acid, tetracosanolpentaenoic acid, cervonic acid, herring acid and mixtures thereof.

Medium-chain triglycerides can be selected from the group consisting of: caprylic/capric glycerides, caprylic/capric glycerides derived from coconut oil or palm seed oil (e.g. Labrafac®, Miglyol® 812, Miglyol® 808 Crodamol GTCC, Softison® 378, Imwitor® 928, Captex® 300, Captex® 355), tricaprylin (Miglyol® 808), and mixtures thereof.

Medium-chain fatty acids can be selected from the group consisting of: caproic acid, caprylic acid, capric acid, and mixtures thereof.

In the context of the present invention, the “second lipid fraction” are mono- and diacylglycerols of medium-chain (C8÷C12) and long-chain (C>12) saturated and unsaturated fatty acids, as well as derivatives of mono- and diacylglycerols of medium-chain (C8÷C12) and long-chain (C>12) saturated and unsaturated fatty acids containing polyethylene glycol in their structure.

Preferred glycerol and propylene glycol esters of medium to long chain fatty acids, which can be used in the present invention include: caprylate/caprate diglycerides, glyceryl monooleate, glyceryl ricinoleate, glyceryl laurate, glyceryl dilaurate, glyceryl dioleate, glyceryl mono/dioleate, glyceryl monocaprylate (Imwitor® 988, Imwitor® 308, Campul 808G EP/NF; Campul MCM-8), glycerol monocaprylocaprate (Imwitor® 742; Campul MCM EP/NF, Campul MCM NF), glyceryl cocoate (Imwitor® 928), mono- and diacetylated monoglycerides, propylene glycol monocaprylate (Capryol PGMC) propylene glycol caprylate/caprate (Labrafac® PC), propylene glycol dicaprylate/dicaprate (Miglyol® 840), propylene glycol monolaurate (Capmul PG-12 NEP/NF), propylene glycol ricinoleate, propylene glycol monooleate (Peceol), glyceryl monolinoleate (Maisine CC), propylene glycol dicaprylate/dicaprate, propylene glycol dioctanoate, glyceryl monoricinoleate (Softigen 701) and mixtures thereof.

The components of the composition of the invention, after mixing in an aqueous medium, spontaneously form essentially spherical nanoparticles outer layer of which is a surfactant and the centre (inner layer) is a mixture of lipids in which a cannabinoid or cannabinoid extract is dissolved. The surfactant that forms the outer layer is anchored to the inner, core lipid layer with its lipophilic part (a fragment of the surfactant molecule constituting a fatty acid residue). At the same time, lipid 2, which plays a key role in the structure of the nanoparticle of the invention, participates in the interactions between its hydrophilic outer layer and the inner hydrophobic core, allowing to obtain the favourable properties of nanoparticles disclosed in the application. The components of the composition, after mixing in an aqueous medium, spontaneously form nanoparticles of the invention, the approximate composition of which changes as follows upon approaching the nanoparticle centre: the environment, being an aqueous solution, further a surfactant, which dominates the outer layer of the nanoparticle, further a mixture of lipids with the active substance (Lipid 1+Lipid 2+Cannabinoids), which are the core of the nanoparticle, where the Lipid 2 component most likely dominates the outer layer of the core, supporting the surfactant.

Preferably, the dispersed phase of the monodisperse system of the invention is formed by particles smaller than 180 nm.

Preferably, the monodisperse system of the invention has: transparency above 90%, monodispersity below (PdI) 0.250, maintains nanoemulsion stability when diluted up to 10,000× with water or an aqueous solution and/or maintains nanoemulsion stability at a pH ranging from pH=8 to below pH<3.

The main technical effect of the developed composition enabling the dissolution of cannabinoids in an aqueous solution is:
high transparency of nanoemulsions when added to an aqueous solution, above 85%;
very short time of forming a homogeneous dispersion system after adding to an aqueous solution, less than 60 s;
no need for extensive mixing, mixing below 2500 RPM and even below 150 RPM;
obtaining nanoparticles of a small size, size below 180 nm hydrodynamic diameter;
obtaining a monodisperse nanosystem, monodispersity (PdI) below 0.250;
high stability of nanoemulsions at very high dilutions, dilution up to 10,000×;
high resistance to a wide range of pH from pH=8 to pH<3, and to final product preservation processes (e.g. pasteurization, cold filtering, UV radiation).

The following symbols are used in this description:
T80—a mixture of polyoxyethylene derivatives of sorbitan and oleic acid, polysorbate 80, Tween® 80 surfactant,
MCT—medium chain triglycerides (C8÷C12)
MCM—medium chain mono-diglycerides, mainly caprylic (C8) and capric (decanoic, 010) acids (mono-diglyceride of medium chain fatty acids),
CBD—cannabidiol, isolate with a purity of 95%.

Study of the Prior Art Compositions.

The prior art compositions have been tested based on U.S. Patent 20200037638i. The contents of the formulations are briefly described in the patent specification as a mixture of MCT and/or LCT (long-chain triacylglycerides) and a surfactant and a cannabinoid extract. Nanoemulsions produced within 2 hours were described in the document, while the mixing speed was not given. This is crucial since the nanoemulsion quality increases with increasing mixing intensity, but the manufacturing process time it lengthened at the industrial scale. In addition, the results describe only the particle size without reference to the particle size distribution. No information on the quality of the emulsion has been identified, namely whether a polydisperse or monodisperse system was obtained. Polydisperse systems tend to aggregate, flocculate and sediment. For the above reasons, it was concluded that experiments should be performed for research purposes, based on the patent description US 20200037638.

The following products were used to study the prior art formulations:
MCT—medium-chain triacylglyceride, using a commercially available product Crodamol™ GTCC from CRODA,
T80—polysorbate 80, using a commercially available product Tween 80 from CRODA,
CBD—cannabidiol, a 95% purity isolate purchased from Kannastar.

TABLE 1
Study of the competitive compositions.

| | MCT | T80 | CBD | Homogeneous | Dispersion | Transmittance | D_{H} | |
|---|---|---|---|---|---|---|---|---|
| No. | % | % | % | system | time [sec] | [%] 100× | [nm] | Pdl |
| 1 | 0 | 90 | 10 | YES | 198 | 16.6 | 157.2 | 0.383 |
| 2 | 10 | 80 | 10 | YES | 382 | 0.55 | n/d | n/d |
| 3 | 20 | 70 | 10 | YES | 840 | 0.35 | n/d | n/d |
| 4 | 30 | 60 | 10 | YES | 573 | 33.8 | 120.3 | 0.165 |
| 5 | 40 | 50 | 10 | YES | 600 | 1.32 | 156.5 | 0.195 |
| 6 | 50 | 40 | 10 | YES | 620 | 0.11 | 191.5 | 0.268 |
| 7 | 60 | 30 | 10 | NO | n/d | n/d | n/d | n/d |
| 8 | 70 | 20 | 10 | NO | n/d | n/d | n/d | n/d |
| 9 | 80 | 10 | 10 | NO | n/d | n/d | n/d | n/d |
| 10 | 0 | 80 | 20 | YES | >1800 | 0.14 | n/d | n/d |
| 11 | 10 | 70 | 20 | YES | 390 | 0.13 | 372.7 | 0.661 |
| 12 | 20 | 60 | 20 | YES | 568 | 0.11 | 271.9 | 0.466 |
| 13 | 30 | 50 | 20 | YES | >1800 | 0.17 | 193.1 | 0.528 |
| 14 | 40 | 40 | 20 | YES | >1800 | 0.16 | 218.6 | 0.518 |
| 15 | 50 | 30 | 20 | YES | >1800 | 38.3 | n/d | n/d |
| 16 | 60 | 20 | 20 | NO | n/d | n/d | n/d | n/d |
| 17 | 70 | 10 | 20 | NO | n/d | n/d | n/d | n/d |
| 18 | 80 | 0 | 20 | NO | n/d | n/d | n/d | n/d |

The table shows the percentages by weight.
n/d—not determined (due to the sample heterogeneity and/or the polydisperse sample not suitable for testing).

Summary of the study of the known formulations presented in Table 1: the study shows that in all cases the dispersion time was much longer than 60 s; transmittance of 85% or more was not achieved in any of the cases; a large part of the compositions were heterogeneous, which prevented their further testing; homogeneous nanoemulsions in the case of compositions 2 and 3 were marked as “n/d” for D_{H} and PdI, because they had a very high polydispersity (>0.500), therefore, in such cases, the samples do not meet the qualitative minimum for correct measurement; the values of PdI are greater than 0.500, and for the D_{H} parameter are greater than 250 nm.

In order to develop an improved composition containing cannabinoids that allows these compounds to be dissolved in aqueous solutions, a number of experiments have been carried out to select the optimal ingredients in terms of dissolution efficiency and amounts used, unexpectedly resulting in the composition of the invention constituting a stable nanoemulsion of water-insoluble cannabinoids that is characterized at the same time by: high transparency, short time of forming a homogeneous dispersion system of cannabinoids, no need for extensive mixing to prepare the dispersion system, obtaining nanoparticles with a small hydrodynamic diameter, monodispersity of the nanosystem and high dilution resistance in aqueous solutions. In addition, the developed formulations were also tested for stability at low pH (below 3) and resistance to preservation (pasteurization).

### GENERAL PROCEDURE FOR THE PREPARATION OF THE COMPOSITIONS OF THE INVENTION

All ingredients of the composition were weighed out into a common vessel. The contents of the vessel were mixed for 30 min at room temperature. Alternatively, the entire mixture may be heated to accelerate the dissolution of the ingredients and then mixed to homogenize the mixture.

Example Procedure for Preparing a Composition Containing 10% by Weight of CBD:

The composition was prepared by weighing out into one vessel 0.460 mg of surfactant (T80), 220 mg of medium chain triacylglyceride (MCT), 220 mg of medium chain mono-diacylglyceride (MCM) and 100 mg of cannabinoid extract (CBD). The mixture was mixed for 30 min with a stir bar at 300 RPM until uniform at room temperature, or for a shorter time at higher temperature (e.g. 60° C.).

Procedure for the Preparation of Nanoemulsions.

Basic dilution—hundredfold (100×) was obtained by introducing 100 microliters (4) of the finished composition by dropping, in a short time (<10 seconds), into a glass vial with a stirring bar containing 10 millilitres (mL) of water. The contents of the glass vial were mixed with rotation up to 150 RPM. The dispersion time was measured until a homogeneous system was obtained.

A 200-fold (200×) dilution was obtained by diluting 1:1 (v/v) the previously prepared nanoemulsion of the 100-fold dilution of the composition with water.

Methodology of Conducted Research
dispersion time was measured in seconds (t, sec) from the moment of adding the last drop of the mixture to water until complete dissolution,
transmittance of nanoemulsions was measured in percent (% T) at a wavelength of λ=600 nm using a Merck® spectrometer model Pharo 300,
hydrodynamic diameter of the nanoparticles was given in nanometers (D_{H}, nm) and measured using a Zetasizer Nano-ZS device from Malvern,
polydispersity of the particles after dispersion in water (polydispersity index) was expressed as a PdI value and measured using a Zetasizer Nano-ZS device from Malvern.

Development of a Self-Emulsifying Cannabinoid Composition of the Invention

The composition that is the subject of the invention contains 4 components:

| 1 | 2 | 3 | 4 |
|---|---|---|---|
| Surfactant | Lipid 1 | Lipid 2 | Cannabinoid |

wherein:
The surfactant (T80) is a polyoxyethylene derivative of sorbitan and oleic acid (Polysorbate 80, Polyoxyethylene Sorbitan Monooleate, Tween™ 80), commercially available and offered by many manufacturers. Tween™ 80 was used in the experiment.
The Lipid 1 (MCT) is a medium chain triglyceride (C8-012 acid mixture) commercially available and offered by many manufacturers. Crodamol™ GTCC was used in the experiment.
The Lipid 2 (MCM) is a mono-diglyceride of medium chain fatty acids, mainly caprylic (C8) and capric (decanoic, 010), commercially available and offered by many manufacturers. 101 OLEOCHEMICAL—Imwitor® 988 was used in the experiment.
The cannabinoid (CBD), possibly as a component of a cannabinoid extract, means extracts and isolates that are commercially available products and are offered by many manufacturers. In the application, most of the testing was performed using cannabidiol, 95% purity isolate purchased from Kannastar.

The content of the composition and the proportion of ingredients are % by weight (wt./wt./wt./wt.). The embodiments of the compositions of the invention refer to the following content ranges of the individual components:
T80: 30-50%,
MCT: 20-35%,
MCM: 15-30%,
CBD: 0-20%.

TABLE 2
Embodiments of the compositions of the invention

| | T80 | MCT | MCM | CBD | Homogeneous | Dispersion | Transmittance | Transmittance | D_{H} | |
|---|---|---|---|---|---|---|---|---|---|---|
| No. | % | % | % | % | system | time [sec] | [%] 100× | [%] 200×* | [nm] | Pdl |
| 19 | 37.0 | 35.0 | 20.1 | 7.9 | YES | 20 | 36.1% | 58.8% | 113.3 | 0.219 |
| 20 | 30.0 | 20.0 | 30.0 | 20.0 | YES | 29 | 0.13% | 0.86% | 154.1 | 0.136 |
| 21 | 36.1 | 24.6 | 19.3 | 20.0 | YES | 67 | 0.12% | 0.59% | 200.0 | 0.278 |
| 22 | 36.1 | 24.6 | 19.3 | 20.0 | YES | 70 | 0.12% | 0.44% | 190.9 | 0.254 |
| 23 | 30.0 | 30.0 | 25.0 | 15.0 | YES | 10 | 0.82% | 8.68% | 188.6 | 0.256 |
| 24 | 36.3 | 20.0 | 28.9 | 14.8 | YES | 15 | 20.4% | 44.6% | 135.6 | 0.246 |
| 25 | 30.0 | 30.0 | 25.0 | 15.0 | YES | 30 | 0.43% | 5.87% | 150.3 | 0.147 |
| 26 | 30.0 | 35.0 | 15.0 | 20.0 | YES | 86 | 0.11% | 0.13% | 262.0 | 0.472 |
| 27 | 42.1 | 27.7 | 22.7 | 7.5 | YES | 20 | 76% | 86.1% | 71.3 | 0.209 |
| 28 | 44.1 | 20.0 | 30.0 | 5.9 | YES | 30 | 96.3% | 97.9% | 32.5 | 0.205 |
| 29 | 42.7 | 20.0 | 24.7 | 12.5 | YES | 15 | 74.3% | 85.9% | 66.3 | 0.177 |
| 30 | 30.9 | 35.0 | 30.0 | 4.1 | YES | 15 | 35.2% | 58.9% | 115.7 | 0.188 |
| 31 | 37.2 | 24.8 | 30.0 | 8.0 | YES | 15 | 59.6% | 77.1% | 104.0 | 0.274 |
| 32 | 43.1 | 30.0 | 15.0 | 11.9 | YES | 50 | 31.3% | 55.5% | 106.2 | 0.143 |
| 33 | 42.2 | 31.1 | 26.7 | 0.0 | YES | 22 | 91.9% | 95.9% | 50.9 | 0.238 |
| 34 | 48.3 | 20.0 | 15.0 | 16.7 | YES | 80 | 15.4% | 38.9% | 124.2 | 0.214 |
| 35 | 42.2 | 31.1 | 26.7 | 0.0 | YES | 30 | 86.3% | 93% | 62.6 | 0.255 |
| 36 | 50.0 | 35.0 | 15.0 | 0.0 | YES | 250 | 4.91% | 22% | 266.5 | 0.293 |
| 37 | 50.0 | 25.2 | 20.2 | 4.6 | YES | 30 | 95% | 97.3% | 35.8 | 0.194 |
| 38 | 36.4 | 34.3 | 15.0 | 14.3 | YES | 70 | 0.2% | 3.07% | 265.3 | 0.266 |
| 39 | 50.0 | 20.0 | 30.0 | 0.0 | YES | 25 | 98.7% | 99.5% | 23.8 | 0.198 |
| 40 | 37.0 | 35.0 | 20.1 | 7.9 | YES | 25 | 11% | 33% | 139.5 | 0.217 |
| 41 | 37.2 | 24.8 | 30.0 | 8.0 | YES | 20 | 57.3% | 75.3% | 90.5 | 0.186 |
| 42 | 50.0 | 24.4 | 15.0 | 10.6 | YES | 50 | 77.9% | 87.7% | 69.6 | 0.193 |

*200-fold (200×) dilution was performed as a control parameter for the quality of nanoemulsions and the performance of self-emulsifying compositions in order to better characterize the systems by transmittance (% T).

Study of Selected Examples of Compositions

Examples of compositions with different contents of cannabinoid extracts were also studied.

The following systems have been found as particularly preferable for the different formulation applications (T80:MCT:MCM:CBD, wt./wt./wt./wt.) as listed below:
0% CBD (44.6:25.4:30.0:0),
5% CBD (44.3:26.9:23.7:5),
10% CBD (46.0:22.0:22.0:10),
15% CBD (43.7:20.0:21.3:15),
20% CBD (37.3:20.0:22.7:20).

TABLE 3
Properties of compositions with different contents of cannabinoid extracts.

| | | | Dispersion | Transmittance | Transmittance | D_{H} | |
|---|---|---|---|---|---|---|---|
| # | Name | | time [sec] | [%] 100× | [%] 200×* | [nm] | Pdl |
| 1 | 0% | CBD | 15 | 98.1% | 99.1% | 23.9 | 0.162 |
| 2 | 0% | CBD | 28 | 97.2% | 99.4% | 24.0 | 0.146 |
| 3 | 0% | CBD | 25 | 97.9% | 99.5% | 22.8 | 0.103 |
| 4 | 5% | CBD | 29 | 94.5% | 97.3% | 37.7 | 0.177 |
| 5 | 5% | CBD | 29 | 94.3% | 96.9% | 38.0 | 0.192 |
| 6 | 5% | CBD | 27 | 95.3% | 96.6% | 39.2 | 0.205 |
| 7 | 10% | CBD | 27 | 93.2% | 95.0% | 46.6 | 0.175 |
| 8 | 10% | CBD | 23 | 92.2% | 95.3% | 44.6 | 0.168 |
| 9 | 10% | CBD | 27 | 91.3% | 95.1% | 45.6 | 0.179 |
| 10 | 15% | CBD | 39 | 50.6% | 71.1% | 86.5 | 0.181 |
| 11 | 15% | CBD | 44 | 51.6% | 70.9% | 95.5 | 0.229 |
| 12 | 15% | CBD | 63 | 52.8% | 71.4% | 87.3 | 0.172 |
| 13 | 20% | CBD | 62 | 0.4% | 4.7% | 148.9 | 0.198 |
| 14 | 20% | CBD | 52 | 0.2% | 2.6% | 154.1 | 0.166 |
| 15 | 20% | CBD | 72 | 0.2% | 1.6% | 153.2 | 0.147 |

Average results (n = 3)

| | | | Dispersion | Transmittance | Transmittance | D_{H} | |
|---|---|---|---|---|---|---|---|
| No. | Name | | time [sec] | [%] 100× | [%] 200×* | [nm] | Pdl |
| 43 | 0% | CBD | 23 ± 7 | 97.7 ± 0.5% | 99.3 ± 0.2% | 23.6 ± 0.7 | 0.137 ± 0.031 |
| 44 | 5% | CBD | 28 ± 1 | 94.7 ± 0.5% | 96.9 ± 0.4% | 38.3 ± 0.8 | 0.191 ± 0.014 |
| 45 | 10% | CBD | 26 ± 2 | 92.2 ± 1.0% | 95.1 ± 0.2% | 45.6 ± 1.0 | 0.174 ± 0.006 |
| 46 | 15% | CBD | 49 ± 13 | 51.7 ± 1.1% | 71.1 ± 0.3% | 89.8 ± 5.0 | 0.194 ± 0.031 |
| 47 | 20% | CBD | 62 ± 10 | 0.3 ± 0.1% | 3.0 ± 1.6% | 152.1 ± 2.8 | 0.170 ± 0.026 |

All variants of the self-emulsifying compositions and dispersions systems were prepared in triplicate and homogeneous samples were obtained.

The study, the results of which are presented in Table 3 (above), demonstrated that the dispersion time for the composition (43-46) did not exceed 60 seconds, while with the 20% content of cannabinoids for the sample (47), the dispersion time was just over 60 seconds. The transmittance above 90% was achieved for the composition containing up to 10% of CBD (45), but at higher CBD concentrations, i.e. above 10% (46-47), the minimum transmittance value, i.e. 85%, was not reached; all compositions presented in Table 3 are homogeneous and have very low PdI values <0.200, which proves a very good quality of nanoemulsions (high emulsion stability); the obtained hydrodynamic diameter of nanoparticles is very low for all compositions and is in the range of D_{H}<180 nm; moreover, up to 15% CBD content (inclusive) it is less than 100 nm.

Study of the Compositions Using a Variety of Cannabinoids.

A significant advantage of the self-emulsifying compositions of the invention is that these compositions are universal and perform well with various hydrophobic compounds, therefore experiments have been carried out using a variety of cannabinoids. Variants of compositions were tested, whereas cannabinoids were used as pure compounds, i.e. cannabidiol (CBD), tetrahydrocannabinol (THC), a mixture of various naturally occurring cannabinoids (full spectrum, abbreviated as “MIX”), various extracts with lower cannabinoid content (designated as “Extracts type . . . ”), as well as hemp oil with a trace amount of cannabinoids and characterized by a high content of unsaturated fatty acids.

All compositions were based on one, recognized as the model, self-emulsifying composition containing 10% of the raw material content—cannabinoid. (T80:MCT:MCM:Cannabinoid, wt./wt./wt./wt.).

The research was conducted with the use of:
CBD—isolate with a purity of ≥95%,
THC—isolate with a purity of ≥95%,
MIX—a mixture of naturally occurring cannabinoids and phytosterols, terpenes, cannabinoid content approx. 95%,
Extract type 1—a mixture of naturally occurring cannabinoids with a content of 70.66% CBD, 4.36% THC, 2.18% CBG,
Extract type 2—a mixture of naturally occurring cannabinoids with a content of 29.79% CBDA, 24.31% CBD, 1.26% THC, 0.60% THCA,
Extract type 3—a mixture of naturally occurring cannabinoids with a content of 25.17% CBD, 18.79% CBDA, 1.30% THC, 0.14% THCA, 1.84% CBC, 0.89% CBG, 0.49% CBGA, 0.22% CBDV, 0.05% CBL, 0.04% CBN, 0.04% delta-8-THC,
Hemp oil—oil obtained from hemp seeds (Cannabis sativia) containing approx. 54% of linoleic acid, 15% of α-linolenic acid, 4% of Y-linolenic acid, 13% of oleic acid and 14% of other fatty acids.

TABLE 4
Properties of compositions using various cannabinoids.

| | | Dispersion | Transmittance | Transmittance | D_{H} | |
|---|---|---|---|---|---|---|
| # | Name | time [sec] | [%] 100× | [%] 200×* | [nm] | Pdl |
| 1 | 10% MIX | 28 | 90.9% | 93.6% | 49.0 | 0.184 |
| 2 | 10% MIX | 23 | 86.4% | 92.2% | 52.7 | 0.226 |
| 3 | 10% MIX | 27 | 84.6% | 92.0% | 51.3 | 0.200 |
| 4 | 10% THC | 32 | 95.5% | 97.7% | 31.0 | 0.183 |
| 5 | 10% THC | 20 | 96.9% | 98.5% | 32.5 | 0.223 |
| 6 | 10% THC | 38 | 96.6% | 98.0% | 32.9 | 0.235 |
| 7 | 10% Extract | 19 | 96.1% | 97.4% | 33.4 | 0.234 |
| | type 1 | | | | | |
| 8 | 10% Extract | 19 | 95.9% | 98.2% | 34.3 | 0.224 |
| | type 1 | | | | | |
| 9 | 10% Extract | 30 | 95.3% | 96.9% | 34.8 | 0.231 |
| | type 1 | | | | | |
| 10 | 10% Extract | 19 | 94.4% | 99.8% | 23.2 | 0.207 |
| | type 2 | | | | | |
| 11 | 10% Extract | 20 | 94.3% | 100.3% | 22.9 | 0.238 |
| | type 2 | | | | | |
| 12 | 10% Extract | 23 | 93.1% | 100.5% | 22.1 | 0.202 |
| | type 2 | | | | | |
| 13 | 10% Extract | 22 | 89.9% | 98.6% | 37.9 | 0.174 |
| | type 3 | | | | | |
| 14 | 10% Extract | 23 | 93.1% | 96.2% | 37.2 | 0.179 |
| | type 3 | | | | | |
| 15 | 10% Extract | 13.5 | 93.2% | 98.1% | 37.2 | 0.183 |
| | type 3 | | | | | |
| 16 | 10% Hemp oil | 35 | 90.6% | 94.4% | 62.0 | 0.284 |
| 17 | 10% Hemp oil | 33 | 92.7% | 95.6% | 54.5 | 0.283 |
| 18 | 10% Hemp oil | 28 | 90.9% | 95.0% | 59.7 | 0.276 |

Average results (n = 3)

| | | Dispersion | Transmittance | Transmittance | D_{H} | |
|---|---|---|---|---|---|---|
| No. | Name | time [sec] | [%] 100× | [%] 200×* | [nm] | Pdl |
| 45 | CBD | 26 ± 2 | 92.2 ± 1.0% | 95.1 ± 0.2% | 45.6 ± 1.0 | 0.174 ± 0.006 |
| 48 | MIX | 26 ± 3 | 87.3 ± 3.2% | 92.6 ± 0.9% | 51.0 ± 1.9 | 0.203 ± 0.021 |
| 49 | THC | 30 ± 9 | 96.3 ± 0.7% | 98.1 ± 0.4% | 32.1 ± 1.0 | 0.214 ± 0.027 |
| 50 | Extract type 1 | 23 ± 6 | 95.8 ± 0.4% | 97.5 ± 0.7% | 34.2 ± 0.7 | 0.230 ± 0.005 |
| 51 | Extract type 2 | 21 ± 2 | 93.9 ± 0.7% | 100.2 ± 0.4% | 22.7 ± 0.6 | 0.216 ± 0.020 |
| 52 | Extract type 3 | 20 ± 5 | 92.1 ± 1.9% | 97.6 ± 1.3% | 37.4 ± 0.4 | 0.179 ± 0.005 |
| 53 | Hemp oil | 32 ± 4 | 91.1 ± 1.1% | 95.0 ± 0.6% | 58.7 ± 3.8 | 0.281 ± 0.004 |

The conducted study demonstrates that the dispersion time for all formulations (45, 48÷53) did not exceed 60 s, the self-emulsifying compositions have similar characteristics regardless of the hemp raw material and the degree of its purification, transmittance for 100× (100-fold) dilution in water in most cases exceeded 90%, with the exception of raw material containing full spectrum of cannabinoids, as well as terpenes and phytosterols (48), the presence of which slightly reduced the transmittance to the value of 87%, all compositions, regardless of the raw material used, had a small nanoparticle hydrodynamic diameter of D_{H}<60 nm. All compositions, regardless of the raw material used, were monodisperse maintaining PdI<0.250 with the exception of sample 53 which had PdI value below 0.300.

Study on the Selection of the Surfactant Contained in the Composition.
The properties of variants of the compositions containing the above-defined particularly preferable proportions of the remaining ingredients determined for 10% CBD model formulation, in which various surfactants (surfactant:MCT:MCM:CBD, wt./wt./wt./wt.) were used, was also investigated.

The study was carried out with the use of the following surfactants from appropriate groups:
monoacylated derivatives of polyoxyethylene sorbitan—there are many different derivatives of this class of surfactants, for which the abbreviation Tween™ is commonly used and depending on the acyl residue of the respective fatty acid, Tween 20, 40, 60, 80 and 85 are distinguished. In the application, two of them were used in addition to Tween™ 80:
   polyoxyethylene sorbitan monolaurate as “Tween 20” (Polysorbate 20, Polyoxyethylene 20 sorbitan monolaurate), commercially available and offered by many manufacturers. Tween™ 20 from CRODA was used in the experiment;
   polyoxyethylene sorbitan monostearate as “Tween 60” (Polysorbate 60, Polyoxyethylene 60 sorbitan monostearate), commercially available and offered by many manufacturers. Tween™ 60 from CRODA was used in the experiment;
ethoxylated oil derivatives as “Etocas”—a surfactant based on polyoxyethylene (35) castor oil (PEG-35 castor oil, Polyoxyl 35 Castor Oil), commercially available and offered by many manufacturers. Etocas™ 35 from CRODA was used in the experiment;
hydrogenated ethoxylated derivatives of oils as “Croduret”—a surfactant based on polyoxyl 40 hydrogenated castor oil (PEG-40 hydrogenated castor oil), commercially available and offered by many manufacturers. Croduret™ 50 from CRODA was used in the experiment;
acyl polyglyceryl derivatives, as “Plurol”—polyglyceryl-3 dioleate, commercially available surfactants offered by many manufacturers. Plurol® Oleique from GATTEFOSE was used in the experiment.

TABLE 5
Surfactant selection study for the self-emulsifying composition.

| | | Dispersion | Transmittance | Transmittance | D_{H} | |
|---|---|---|---|---|---|---|
| # | Name | time [sec] | [%] 100× | [%] 200×* | [nm] | Pdl |
| 1 | Tween 20 | 44 | 29.7% | 65.3% | 110.8 | 0.133 |
| 2 | Tween 20 | 55 | 36.3% | 58.3% | 106.8 | 0.131 |
| 3 | Tween 20 | 80 | 40.2% | 61.3% | 109.0 | 0.155 |
| 4 | Tween 60 | 58 | 56.3% | 76.5% | 97.2 | 0.203 |
| 5 | Tween 60 | 32 | 63.5% | 81.0% | 95.3 | 0.211 |
| 6 | Tween 60 | 28 | 56.4% | 76.0% | 118.2 | 0.266 |
| 7 | Etocas | 515 | 95.8% | 100.7% | 98.7 | 0.188 |
| 8 | Etocas | 420 | 95.8% | 101.1% | 28.5 | 0.169 |
| 9 | Etocas | 305 | 96.5% | 101.6% | 37.4 | 0.237 |
| 10 | Croduret | 548 | 96.3% | 100.8% | 27.1 | 0.081 |
| 11 | Croduret | 607 | 97.1% | 98.9% | 28.0 | 0.101 |
| 12 | Croduret | 364 | 98.1% | 101.8% | 28.5 | 0.100 |
| 13 | Plurol | heterogeneous | n/d | n/d | n/d | n/d |
| 14 | Plurol | system | | | | |
| 15 | Plurol | | | | | |

Average results (n = 3)

| | | Dispersion | Transmittance | Transmittance | D_{H} | |
|---|---|---|---|---|---|---|
| No. | Name | time [sec] | [%] 100× | [%] 200×* | [nm] | Pdl |
| 45 | Tween 80 | 26 ± 2 | 92.2 ± 1.0% | 95.1 ± 0.2% | 45.6 ± 1.0 | 0.174 ± 0.006 |
| 54 | Tween 20 | 60 ± 18 | 35.4 ± 5.3% | 61.6 ± 3.5% | 108.9 ± 2.0 | 0.140 ± 0.013 |
| 55 | Tween 60 | 39 ± 16 | 58.7 ± 4.1% | 77.8 ± 2.8% | 103.6 ± 12.7 | 0.227 ± 0.034 |
| 56 | Etocas 35 | 413 ± 105 | 96.0 ± 0.4% | 101.1 ± 0.5% | 54.9 ± 38.2 | 0.198 ± 0.035 |
| 57 | Croduret | 506 ± 127 | 97.2 ± 0.9% | 100.5 ± 1.5% | 27.9 ± 0.7 | 0.094 ± 0.011 |
| 58 | Plurol | n/d | n/d | n/d | n/d | n/d |

n/d—not determined (due to the sample heterogeneity and/or the polydisperse sample not suitable for testing).

The results of the study show that the compositions containing Tween™ 80 (45) have the best dispersion time, very good nanoparticle size parameters, are characterized by monodispersity, while the alternative Tween™ 60 (55) is characterized by much lower transmittance, and the compositions containing Etocas (56) and Croduret (57) are characterized by very high transmittance, while they disperse worse and longer than the composition containing Tween™ 80 (45).

Therefore, in the preferred embodiment of the compositions of the invention, Tween™ 80 is used as the surfactant.

Study on the Selection of the Lipid Component (Lipid 1) Contained in the Composition.

A parallel study was also carried out on the selection of the lipid component Lipid 1, which in the preferred composition of the invention is a medium-chain triacylglyceride (MCT) with a mixture of acids of C8÷C12 length. Medium-chain triacylglycerides are predominantly esters with caprylic (octanoic, C8:0) and capric (decanoic, 010:0) acids. MCTs are commercially available and offered by many manufacturers under various trade names, including: Crodamol GTCC triacylglyceride from CRODA, Labrafac lipophile from GATEFOSSE, Miglyol 808 from 101 OLEOCHEMICAL, Miglyol 812 N from 101 OLEOCHEMICAL, Imwitor® 928 from 101 OLEOCHEMICAL, Captex® 300 from ABITEC or Captex® 355 from ABITEC. The first two were tested, forming compositions identical in terms of parameters.

The formulations containing the preferred ingredient ratios specified above for 10% CBD model formulation content with different lipids instead of MCT (T80:Lipid 1:MCM:CBD, wt./wt./wt./wt.) were also investigated.

The following groups of compounds were used in the study as the lipid component Lipid 1:
hemp oil—oil obtained from hemp seeds (Cannabis sativia) containing approximately 54% of linoleic acid, 15% of α-linolenic acid, 4% of γ-linolenic acid, 13% of oleic acid and 14% of other fatty acids. The compound used is classified as a long chain triacylglyceride (LCT) with a mixture of C14÷C26 acids,
fatty acid as “FA”—oleic acid was chosen as an example of this group of compounds, which is an example of a long-chain free fatty acid, which has 18 carbon atoms and one double bond (018:1). Fatty acids are commercially available and offered by many manufacturers. Oleic acid with a purity of >95% purchased at Sigma-Aldrich was used in the experiment.
triacylglyceride as “TAG”—natural oil was chosen as an example of this group of compounds, which mainly contains various triacylglycerides composed mostly of oleic acid derivatives, but also palmitic acid, linoleic acid and other fatty acids. A commercial food grade oil, sold as olive oil, which also contains small amounts (<1%) of squalene, phenolic compounds and vitamin E, was used in the experiment.

TABLE 6
Lipid component (Lipid 1) selection study for the self-emulsifying composition.

| | | Dispersion | | | | |
|---|---|---|---|---|---|---|
| | | time | Transmittance | Transmittance | D_{H} | |
| # | Name | [sec] | [%] 100× | [%] 200×* | [nm] | Pdl |
| 1 | hemp oil | 14 | 55.2% | 74.1% | 53.4 | 0.387 |
| 2 | hemp oil | 21 | 69.1% | 80.1% | 50.4 | 0.271 |
| 3 | hemp oil | 21 | 71.2% | 83.5% | 54.4 | 0.301 |
| 4 | FA | heterogeneous | n/d | n/d | n/d | n/d |
| 5 | FA | system | | | | |
| 6 | FA | | | | | |
| 7 | TAG | heterogeneous | n/d | n/d | n/d | n/d |
| 8 | TAG | system | | | | |
| 9 | TAG | | | | | |

Average results (n = 3)

| | | Dispersion | Transmittance | Transmittance | D_{H} | |
|---|---|---|---|---|---|---|
| Nr | Name | time [sec] | [%] 100× | [%] 200×* | [nm] | Pdl |
| 45 | MCT | 26 ± 2 | 92.2 ± 1.0% | 95.1 ± 0.2% | 45.6 ± 1.0 | 0.174 ± 0.006 |
| 59 | hemp oil | 19 ± 4 | 65.2 ± 8.7% | 79.2 ± 4.8% | 52.75 ± 2.1 | 0.320 ± 0.060 |
| 60 | FA | n/d | n/d | n/d | n/d | n/d |
| 61 | TAG | n/d | n/d | n/d | n/d | n/d |

n/d—not determined (due to the sample heterogeneity and/or the polydisperse sample not suitable for testing).

The conducted study demonstrates that the compositions containing MCT (45) as the Lipid 1 are the best variant, because they are characterized by a very short dispersion time and high transparency assessed by transmittance measurement. In the case of the variant using hemp oil (59), the system has a lower transmittance and almost twice higher polydispersity coefficient comparing to the optimal composition (45). In the preferred composition of the invention, Lipid 1 is MCT.

Study on the Selection of the Lipid Component (Lipid 2) of the Self-Emulsifying Composition.

A study on the selection of the lipid component Lipid 2, which in the preferred composition of the invention is medium-chain mono-diacylglyceride (MCM) with a mixture of acids of C8÷C12 length was also carried out. Medium-chain mono-diacylglycerides are commercially available and offered by many manufacturers under various trade names, including: Imwitor® 988 (Type I) from 101 OLEOCHEMICAL, Imwitor® 308 (Type II) from 101 OLEOCHEMICAL, Imwitor® 742 (Type I) from 101 OLEOCHEMICAL, Imwitor® 928 from 101 OLEOCHEMICAL, Campul MCM EP/NF (Type I) from ABITEC, Campul 808G EP/NF (Type II) from ABITEC, Campul MCM-8 (Type I) from ABITEC, or Campul MCM NF (Type I) from ABITEC.

TABLE 7
Comparison study for MCM of various manufacturers
and types as the lipid component Lipid 2.

| | | Dispersion | Transmittance | Transmittance | D_{H} | |
|---|---|---|---|---|---|---|
| # | Name | time [sec] | [%] 100× | [%] 200×* | [nm] | Pdl |
| 1 | Imwitor ® 988 | 27 | 93.2% | 95.0% | 46.6 | 0.175 |
| 2 | Imwitor ® 988 | 23 | 92.2% | 95.3% | 44.6 | 0.168 |
| 3 | Imwitor ® 988 | 27 | 91.3% | 95.1% | 45.6 | 0.179 |
| 4 | Campul 808G | 17 | 95.7% | 97.2% | 27.6 | 0.205 |
| 5 | Campul 808G | 18 | 97.0% | 98.5% | 27.9 | 0.182 |
| 6 | Campul 808G | 19 | 97.2% | 97.9% | 27.9 | 0.183 |
| 7 | Capmul MCM | 25 | 94.5% | 96.8% | 37.5 | 0.258 |
| | EP/NE | | | | | |
| 8 | Capmul MCM | 20 | 95.8% | 97.2% | 30.5 | 0.155 |
| | EP/NE | | | | | |
| 9 | Capmul MCM | 37 | 94.9% | 96.9% | 34.9 | 0.174 |
| | EP/NE | | | | | |

Average results (n = 3)

| | | Dispersion | Transmittance | Transmittance | D_{H} | |
|---|---|---|---|---|---|---|
| No. | Name | time [sec] | [%] 100× | [%] 200×* | [nm] | Pdl |
| 45 | Imwitor ® 988 | 26 ± 2 | 92.2 ± 1.0% | 95.10 ± 0.20% | 45.60 ± 1.0 | 0.174 ± 0.006 |
| 62 | Capmul 808G | 18 ± 1 | 96.6 ± 0.8% | 97.87 ± 0.65% | 27.80 ± 0.2 | 0.190 ± 0.013 |
| 63 | Capmul MCM | 27 ± 9 | 95.1 ± 0.7% | 96.97 ± 0.21% | 34.29 ± 3.5 | 0.196 ± 0.055 |
| | EP/NE | | | | | |

Based on Table 7, the compositions containing MCM from different manufacturers as Lipid 2, identical in terms of the contents, form identical nanoparticle systems and are characterized by: similar dispersion time, transmittance, hydrodynamic diameter, and they form monodisperse systems with a similar PdI parameter of just under 0.200.

The formulations containing the preferred ingredient ratios specified above for 10% CBD model formulation content with different lipids instead of MCM (T80:MCT:Lipid 2:CBD, wt./wt./wt./wt.) was also investigated. Compounds that are derivatives of MCM from the following groups were used in the study as the lipid component Lipid 2:
medium chain monoesters with propylene glycol—there are several different derivatives of this group of compounds, which are described in the application as “PGM” (propylene glycol monoester). Three of them were tested in the application:
   caprylic acid monoester (C8:0) with propylene glycol, type I pharmaceutical classification, abbreviated as “PGMC-I” (propylene glycol monocaprylate, PG monocaprylate C8, Type I, NF) under the trade name Capryol PGMC from GATEFOSSE,
   caprylic acid monoester (C8:0) with propylene glycol, type II pharmaceutical classification, abbreviated “PGMC-II” (propylene glycol monocaprylate, PG monocaprylate C8, Type II) under the trade name Capryol 90 from GATEFOSSE,
   lauric acid monoester (C12:0) with propylene glycol, type II pharmaceutical classification, abbreviated “PGML” (propylene glycol monolaureate, Type II) under the trade name Capmul PG-12 NEP/NF from ABITEC,
long chain acid monoesters with glycerol—there are several different derivatives of this group of compounds, which are described in the application as “GM” (glycerol mononoester). Three of them were tested in the application:
   glycerol monoester with oleic acid (C18:1), abbreviated as “GMO” (glyceryl monooleate, Type 40) under the trade name Peceol from GATEFOSSE,
   glycerol monoester with linoleic acid (C18:2), abbreviated as “GML” (glyceryl monolinoleate) under the trade name Maisine CC from GATEFOSSE,
   glycerol monoester with ricinoleic acid (C18:1, —OH), abbreviated as “GMR” (glyceryl monoricinoleate) under the trade name Softigen 701 from IOI OLEOCHEMICAL.

TABLE 8
Lipid component (Lipid 2) selection study for the self-emulsifying composition.

| | | Dispersion | Transmittance | Transmittance | D_{H} | |
|---|---|---|---|---|---|---|
| # | Name | time [sec] | [%] 100× | [%] 200×* | [nm] | Pdl |
| 1 | PGMC-I | 520 | 0.27% | 3.75% | 179.0 | 0.221 |
| 2 | PGMC-I | 470 | 0.28% | 4.05% | 169.5 | 0.193 |
| 3 | PGMC-I | 580 | 0.20% | 2.74% | 183.6 | 0.220 |
| 4 | PGMC-II | 195 | 0.18% | 2.16% | 208.4 | 0.293 |
| 5 | PGMC-II | 209 | 0.34% | 4.99% | 198.5 | 0.270 |
| 6 | PGMC-II | 230 | 0.19% | 2.27% | 203.9 | 0.317 |
| 7 | PGML | >1200 | | | | |
| 8 | PGML | heterogeneous | n/d | n/d | n/d | n/d |
| 9 | PGML | system | | | | |
| 10 | GMO | >1200 | | | | |
| 11 | GMO | heterogeneous | n/d | n/d | n/d | n/d |
| 12 | GMO | system | | | | |
| 13 | GML | >1200 | | | | |
| 14 | GML | heterogeneous | n/d | n/d | n/d | n/d |
| 15 | GML | system | | | | |
| 16 | GMR | 140 | 0.15% | 0.66% | 233.9 | 0.426 |
| 17 | GMR | 160 | 0.13% | 0.31% | 254.0 | 0.447 |
| 18 | GMR | 165 | 0.13% | 0.24% | 285.7 | 0.498 |

Average results (n = 3)

| | | Dispersion | Transmittance | Transmittance | D_{H} | |
|---|---|---|---|---|---|---|
| No. | Name | time [sec] | [%] 100× | [%] 200×* | [nm] | Pdl |
| 45 | MCM | 26 ± 2 | 92.2 ± 1.0% | 95.1 ± 0.2% | 45.6 ± 1.0 | 0.174 ± 0.006 |
| 64 | PGMC-I | 523 ± 55 | 0.25 ± 0.04% | 3.51 ± 0.69% | 177.37 ± 7.2 | 0.211 ± 0.016 |
| 65 | PGMC-II | 211 ± 17 | 0.24 ± 0.09% | 3.14 ± 1.60% | 203.62 ± 5.0 | 0.293 ± 0.023 |
| 66 | PGML | n/d | n/d | n/d | n/d | n/d |
| 67 | GMO | n/d | n/d | n/d | n/d | n/d |
| 68 | GML | n/d | n/d | n/d | n/d | n/d |
| 69 | GMR | 155 ± 13 | 0.14 ± 0.01% | 0.40 ± 0.23% | 257.88 ± 26.1 | 0.457 ± 0.037 |

n/d—not determined (due to the sample heterogeneity and/or the polydisperse sample not suitable for testing).

Table 8 shows that the compositions based on MCM as Lipid 2 are characterized by a very low dispersion time <60 seconds, a high transparency coefficient assessed by transmittance measurement (% T), very desired nanoparticle size parameters while maintaining monodispersity of the system.

In a preferred embodiment of the compositions of the invention, MCM is used as Lipid 2.

Study on the Stability of the Self-Emulsifying Composition During Dilution and Various Dispersion Systems.

The system stability of the preferred composition with 10% and 20% cannabinoid content was confirmed for dispersion systems diluted up to 10000×. The tests were performed on the basis of two compositions with proportions other than the optimal (variants 1 and 2) to demonstrate the versatility of the technology. The contents of the compositions were adjusted to the range tested in the application and the exact values of the studied compositions are shown in Table 9.

TABLE 9
Self-emulsifying compositions tested
at different dispersion dilutions.

| | Abbre- | Surfactant | Lipid 1 | Lipid 2 | Cannabinoid |
|---|---|---|---|---|---|
| Description | viation | T80 | MCT | MCM | CBD |
| Optimal system | CBD 10% | 46% | 22% | 22% | 10% |
| (No. 45) | W-1 | 30% | 30% | 30% | 10% |
| Variant 1 | | | | | |
| Optimal system | CBD 20% | 37.3% | 20% | 22.7% | 20% |
| (No. 47) | W-2 | 30% | 25% | 25% | 20% |
| Variant 2 | | | | | |

In this part of the study, nanoemulsions were made as described in the general description by introducing an appropriate amount of the tested composition. The mixing speed in this case was 500 RPM in each case. The results did not take into account the dispersion time due to very fast dispersion <10 seconds in each case, while the measurement of transmittance (% T), hydrodynamic diameter and polydispersity were assessed at the appropriate dilution of the system.

Three dispersion systems were tested:
water (<0.21 uS) with pH=7.01;
an aqueous solution containing 0.8% by weight of citric acid and 0.08% of sodium benzoate, pH=2.61;
an aqueous solution containing 10% by weight of sucrose, 0.8% by weight of citric acid and 0.08% of sodium benzoate, pH=2.74.

In all tested matrix (water, aqueous solution with the addition of citric acid and sodium benzoate, and aqueous solution with the addition of sugar, citric acid and sodium benzoate), the obtained dispersion system were characterized by very similar parameters, i.e. transparency coefficient assessed by transmittance measurement (% T), nanoparticle hydrodynamic diameter and polydispersity of the system. Table 10 shows only those relating to the use of an aqueous solution containing 0.8% by weight of citric acid and 0.08% of sodium benzoate at pH 2.61 as the dispersion system.

TABLE 10
Nanoemulsion stability study for different
dilutions in aqueous solution at pH = 2.61.

| | | | Transmittance | D_{H} | |
|---|---|---|---|---|---|
| # | Name | Dilution | [%] | [nm] | Pdl |
| 1 | W-1 1000× | 1 000 | 95.5 | 69.8 | 0.115 |
| 2 | W-1 1000× | 1 000 | 95.4 | 72.4 | 0.122 |
| 3 | W-1 1000× | 1 000 | 95.1 | 70.2 | 0.105 |
| 4 | W-1 2500× | 2 500 | 97.7 | 76.4 | 0.142 |
| 5 | W-1 2500× | 2 500 | 97.6 | 73.6 | 0.117 |
| 6 | W-1 2500× | 2 500 | 97.3 | 75.6 | 0.121 |
| 7 | W-1 5000× | 5 000 | 99.1 | 75 | 0.126 |
| 8 | W-1 5000× | 5 000 | 98.9 | 73.9 | 0.124 |
| 9 | W-1 5000× | 5 000 | 99.2 | 69.6 | 0.126 |
| 10 | W-1 10000× | 10 000 | 99.2 | 75.8 | 0.142 |
| 11 | W-1 10000× | 10 000 | 99.2 | 73.6 | 0.135 |
| 12 | W-1 10000× | 10 000 | 99.1 | 122.1 | 0.315 |
| 13 | W-2 1000× | 1 000 | 88.3 | 97.0 | 0.108 |
| 14 | W-2 1000× | 1 000 | 85.1 | 105.2 | 0.128 |
| 15 | W-2 1000× | 1 000 | 87.9 | 99.9 | 0.115 |
| 16 | W-2 2500× | 2 500 | 92.1 | 104.5 | 0.137 |
| 17 | W-2 2500× | 2 500 | 89.6 | 112.3 | 0.141 |
| 18 | W-2 2500× | 2 500 | 90.6 | 108.5 | 0.147 |
| 19 | W-2 5000× | 5 000 | 95.5 | 102.3 | 0.137 |
| 20 | W-2 5000× | 5 000 | 93.4 | 105.9 | 0.151 |
| 21 | W-2 5000× | 5 000 | 91.8 | 153.1 | 0.319 |
| 22 | W-2 10000× | 10 000 | 96.8 | 125.4 | 0.263 |
| 23 | W-2 10000× | 10 000 | 96.1 | 109.6 | 0.152 |
| 24 | W-2 10000× | 10 000 | 95.1 | 119.7 | 0.184 |

Average results (n = 3)

| | | | Transmittance | D_{H} | |
|---|---|---|---|---|---|
| No. | Name | Dilution | [%] | [nm] | Pdl |
| 45 | CBD 10% | 100 | 92.2 ± 1.0% | 45.6 ± 1.0 | 0.174 ± 0.013 |
| 70 | W-1 1000× | 1 000 | 95.3 ± 0.2% | 70.8 ± 1.4 | 0.114 ± 0.009 |
| 71 | W-1 2500× | 2 500 | 97.5 ± 0.2% | 75.2 ± 1.4 | 0.127 ± 0.006 |
| 72 | W-1 5000× | 5 000 | 99.1 ± 0.2% | 72.8 ± 2.9 | 0.125 ± 0.001 |
| 73 | W-1 10000× | 10 000 | 99.2 ± 0.1% | 90.5 ± 27.4 | 0.197 ± 0.102 |
| 47 | CBD 20% | 100 | 0.3 ± 0.1% | 152.1 ± 2.8 | 0.170 ± 0.026 |
| 74 | W-2 1000× | 1 000 | 87.1 ± 0.7% | 100.7 ± 4.2 | 0.117 ± 0.010 |
| 75 | W-2 2500× | 2 500 | 90.8 ± 1.3% | 108.4 ± 3.9 | 0.142 ± 0.005 |
| 76 | W-2 5000× | 5 000 | 93.6 ± 1.9% | 120.4 ± 28.3 | 0.202 ± 0.101 |
| 77 | W-2 10000× | 10 000 | 96.0 ± 0.9% | 118.2 ± 8.0 | 0.200 ± 0.057 |

The compositions of the invention are characterized by dilution stability up to 10,000×. The results summarized in Table 10 show that the tested compositions have a very high transparency factor assessed by transmittance measurement (% T). The dilutions also have no significant effect on the nanoparticle hydrodynamic diameters and the polydispersity of the systems. The results did not include dispersion time due to very fast dispersion <10 seconds.

Study on the Stability of the Active Substance in the Composition and the Stability Test of the Active Substances Under Simulated Preservation Conditions.

An important advantage of the self-emulsifying compositions of the invention is that the compositions are used to prepare oral products based on aqueous solutions, especially pharmaceutical compositions, medical compositions or food products. The above-mentioned products must provide delivery of equal doses of the active substance. For this purpose, the variant of the composition for 1000× dilution was verified for the content of the active substance—cannabidiol (CBD) in the fresh emulsion and after a simulated pasteurization test which was carried out by keeping the dispersion system at 72° C. for 15 min. The active substance content was measured using UHPLC instrument Thermo Scientific Dionex Ultimate 3000 UHPLC⁺ focused, with Thermo Scientific Acclaim RSLC Polar Advantage II column (2.1 mm×100 mm, 2.2 μm) using a two-phase gradient elution: A: 0.1% HCOOH in water and B: 0.1% HCOOH in acetonitrile. The amount of CBD in tested samples was obtained based on a calibration curve made of a standard solution of cannabidiol in methanol, purchased from Merck (CRM, 1.0 mg/mL Cerilliant®, where the regression coefficient of the calibration curve was R²=0.9999 for the limit of detection (LOD) of 1.31 μg/mL and the limit of quantitation (LOQ) of 3.98 μg/mL.

TABLE 11
Evaluation of the active substance content in aqueous solution
at pH = 2.61 before and after simulated pasteurization.

| | | | CBD content in | CBD content in |
|---|---|---|---|---|
| | | | dispersion before | dispersion after |
| # | Name | Dilution | pasteurization [%]* | pasteurization [%]* |
| 1 | W-1 1000x | 1 000 | 98.46 | 96.22 |
| 2 | W-1 1000x | 1 000 | 96.28 | 95.61 |
| 3 | W-1 1000x | 1 000 | 95.75 | 95.87 |
| 4 | W-2 1000x | 1 000 | 97.60 | 98.74 |
| 5 | W-2 1000x | 1 000 | 89.77 | 93.79 |
| 6 | W-2 1000x | 1 000 | 96.43 | 98.85 |

Average results (n = 3)

| | | | CBD content in | CBD content in |
|---|---|---|---|---|
| | | | dispersion before | dispersion after |
| No. | Name | Dilution | pasteurization [%]* | pasteurization [%]* |
| 70 | W-1 1000x | 1 000 | 96.83 ± 1.44 | 95.90 ± 0.31 |
| 74 | W-2 1000x | 1 000 | 94.60 ± 4.22 | 97.13 ± 2.89 |

*% CBD content based on the theoretical content calculated on the basis of the weight

The CBD content in the dispersion for systems 70 and 74 exceeds >90% and in both cases amounts to approx. 95%, which confirms that the reported compositions form stable nanoemulsions and meet the requirement to deliver equal doses of the active substance. The attempt to preserve nanoemulsions by pasteurization in simulated conditions for systems 70 and 74 did not affect the CBD content in these systems, and the differences did not exceed 3% of the active substance content.

Study of the Self-Emulsifying Compositions Stability in Tests of Temperature Transitions (Freeze-Thaw Test)

Commercial products at the distribution stage are exposed to many external factors. One of them is the effect of temperature, especially during transport. Therefore, it is imperative that the product is able to withstand a certain range of temperature fluctuations during transport. The freeze-thaw cycle test is part of the stability study to determine whether the composition parameters remain stable under various conditions. This type of testing is particularly recommended for liquid products. Phase separation can occur in such products, which can have a negative impact on their performance and quality parameters. The freeze-thaw resistance test involves exposing the product to sub-zero temperature (≤−10° C.) for 24 hours, and then incubating it for 24 hours at room temperature. The sample is then placed at a higher temperature (approximately 45° C.) for 24 hours and then again at room temperature for 24 hours. The sample is analyzed for significant changes. One cycle is thus completed. If no significant changes are observed after three cycles of freeze-thaw testing in a row, it can be assured that the product's stability is sufficient for transport. Accordingly, a test of cyclic freeze-thaw, to which food products/raw materials may undergo during transport, was performed on the compositions described below.

Stability testing of self-emulsifying compositions containing 10% and 20% of cannabinoid, respectively, was performed based on four compositions of non-optimal proportions, in order to demonstrate the versatility of the technology. In addition to the compositions described above, two further formulations were included in the tests described below: Variant 3 and Variant 4 for compositions with 10% and 20% cannabinoid content, respectively. The exact content of the tested compositions is shown in Table 12.

TABLE 12
Self-emulsifying compositions tested in the freeze-thaw stability studies.

| | | | Surfactant | Lipid 1 | Lipid 2 | Cannabinoid |
|---|---|---|---|---|---|---|
| No. | Description | Abbreviation | T80 | MCT | MCM | CBD |
| 45 | Optimal system | CBD 10% | 46% | 22% | 22% | 10% |
| 78 | Variant 1 | W-1 | 30% | 30% | 30% | 10% |
| 80 | Variant 3 | W-3 | 50% | 20% | 20% | 10% |
| 47 | Optimal system | CBD 20% | 37.3% | 20% | 22.7% | 20% |
| 79 | Variant 2 | W-2 | 30% | 25% | 25% | 20% |
| 81 | Variant 4 | W-4 | 50% | 15% | 15% | 20% |

The freeze-thaw durability test was carried out on the four compositions described in Table 12, each experiment in triplicate (n=3), where each composition was subjected to three freeze-thaw cycles (1 CYCLE can be described as follows: 24 hours at temp. −21° C., then the samples were transferred for 24 hours to temp. 22° C., further transferred for 24 hours to temp. 40° C., and then for 24 hours to 22° C.). After each cycle, transmittance (T %), size of the nanoparticles (D_{H}), nanoparticle size distribution (PdI) were determined, as well as quantitative analysis of CBD was performed using ultra high performance liquid chromatography (UHPLC-DAD). Additionally, the samples were visually inspected for change of color, odor, phase separation etc. The samples showed no organoleptic changes.

TABLE 13
Values of the quality parameters of the tested compositions in the freeze-thaw tests.

| | | | | Transmittance | D_{H} | | HPLC |
|---|---|---|---|---|---|---|---|
| # | Name | Cycle | Replicate | [%] | [nm] | Pdl | [%] |
| 1 | Variant 1 | 0 (start) | 1 | 97.7 | 76.4 | 0.142 | 99.52 |
| 2 | (Nr 78) | | 2 | 97.6 | 73.6 | 0.117 | 98.68 |
| 3 | | | 3 | 97.3 | 75.6 | 0.121 | 100.78 |
| 4 | | 1^{st} | 1 | 98 | 71.1 | 0.123 | 97.96 |
| 5 | | cycle | 2 | 97.7 | 71.4 | 0.122 | 98.64 |
| 6 | | | 3 | 97.9 | 71.9 | 0.133 | 102.29 |
| 7 | | 2^{nd} | 1 | 98.9 | 79.2 | 0.187 | 97.11 |
| 8 | | cycle | 2 | 98.6 | 76.6 | 0.148 | 97.13 |
| 9 | | | 3 | 98.3 | 72.1 | 0.133 | 100.51 |
| 10 | | 3^{rd} | 1 | 96.3 | 73.7 | 0.134 | 97.03 |
| 11 | | cycle | 2 | 98.5 | 70.1 | 0.135 | 97.22 |
| 12 | | | 3 | 97.9 | 71.7 | 0.125 | 100.12 |
| 13 | Variant 2 | 0 (start) | 1 | 92.1 | 104.5 | 0.137 | 98.75 |
| 14 | (Nr 79) | | 2 | 89.6 | 112.3 | 0.141 | 98.47 |
| 15 | | | 3 | 90.6 | 108.5 | 0.147 | 98.73 |
| 16 | | 1^{st} | 1 | 92.2 | 102.4 | 0.117 | 99.53 |
| 17 | | cycle | 2 | 93.8 | 103.2 | 0.14 | 99.69 |
| 18 | | | 3 | 90.4 | 110.7 | 0.131 | 94.1 |
| 19 | | 2^{nd} | 1 | 93.7 | 104.1 | 0.166 | 99.18 |
| 20 | | cycle | 2 | 93 | 116.9 | 0.225 | 98 |
| 21 | | | 3 | 94.1 | 104.4 | 0.158 | 99.74 |
| 22 | | 3^{rd} | 1 | 94.2 | 100.8 | 0.137 | 99.23 |
| 23 | | cycle | 2 | 91.1 | 109.7 | 0.131 | 98.78 |
| 24 | | | 3 | 94.3 | 101.4 | 0.113 | 99.56 |
| 25 | Variant 3 | 0 (start) | 1 | 98.4 | 49.1 | 0.371 | 99.65 |
| 26 | (Nr 80) | | 2 | 99.1 | 37.8 | 0.26 | 96.93 |
| 27 | | | 3 | 99.7 | 35.5 | 0.213 | 99.12 |
| 28 | | 1^{st} | 1 | 100 | 32.4 | 0.145 | 98.15 |
| 29 | | cycle | 2 | 100.1 | 32.5 | 0.156 | 95.95 |
| 30 | | | 3 | 99.8 | 32.8 | 0.138 | 98.28 |
| 31 | | 2^{nd} | 1 | 99 | 33.6 | 0.176 | 99.32 |
| 32 | | cycle | 2 | 98.6 | 36.5 | 0.241 | 98.17 |
| 33 | | | 3 | 98.3 | 34.3 | 0.208 | 96.72 |
| 34 | | 3^{rd} | 1 | 99.4 | 35.8 | 0.219 | 99.44 |
| 35 | | cycle | 2 | 99.4 | 34.7 | 0.214 | 95.68 |
| 36 | | | 3 | 99.7 | 37.2 | 0.282 | 97.89 |
| 37 | Variant 4 | 0 (start) | 1 | 93.5 | 115.6 | 0.193 | 100.21 |
| 38 | (Nr 81) | | 2 | 93 | 115.1 | 0.157 | 98.97 |
| 39 | | | 3 | 93.5 | 111.7 | 0.152 | 98.73 |
| 40 | | 1^{st} | 1 | 91.4 | 122 | 0.153 | 100.08 |
| 41 | | cycle | 2 | 93.9 | 126.2 | 0.245 | 100.14 |
| 42 | | | 3 | 95 | 112.9 | 0.208 | 99.57 |
| 43 | | 2^{nd} | 1 | 92.5 | 126.1 | 0.219 | 99.34 |
| 44 | | cycle | 2 | 93.2 | 114.1 | 0.161 | 99.41 |
| 45 | | | 3 | 94.5 | 112.3 | 0.183 | 97.05 |
| 46 | | 3^{rd} | 1 | 92 | 118.7 | 0.178 | 99.87 |
| 47 | | cycle | 2 | 93.1 | 117.2 | 0.185 | 98.12 |
| 48 | | | 3 | 93.8 | 112.9 | 0.171 | 99.06 |

Average results (n = 12)

| | | | Repetitions | Transmittance | D_{H} | | HPLC |
|---|---|---|---|---|---|---|---|
| No. | Name | Cycles | per cycle | [%] | [nm] | Pdl | [%] |
| 78 | Variant 1 | 3 | 3 | 97.0 ± 0.7 | 73.62 ± 2.8 | 0.135 ± 0.019 | 98.9 ± 1.7 |
| 79 | Variant 2 | 3 | 3 | 92.4 ± 1.7 | 106.6 ± 5.0 | 0.145 ± 0.029 | 98.6 ± 1.5 |
| 80 | Variant 3 | 3 | 3 | 99.3 ± 0.6 | 36.0 ± 4.5 | 0.219 ± .066 | 97.9 ± 1.4 |
| 81 | Variant 4 | 3 | 3 | 93.3 ± 1.0 | 117.1 ± 5.2 | 0.184 ± .029 | 99.2 ± 0.9 |

UHPLC -% amount of CBD in relation to the nominal value (initial - resulting from the sample weight)

Stability Testing of Self-Emulsifying Compositions in Commercially Available Beverages

Compatibility tests of the developed invention with commercially available beverages were performed based on variant 3 (No. 80). This system demonstrated very high transparency and a short dispersion time. The experiment aimed to determine if a given system could be applied to different categories of beverages. For this purpose, 12 commercial beverages supplemented with a self-emulsifying composition that delivered a dose of 10 mg of CBD per the total volume of the unit drink were tested Various drinks were tested in the experiment: water, flavored drinks, juice beverages, fruit drinks, tonic drinks, lemonade, tea beverage, carbonated and non-carbonated drinks, transparent, colored and lightproof, with and without vitamins added, clarified and unclarified, pasteurized and unpasteurized, sweetened and unsweetened, and even beer up to alcohol content of <5%. Water was used as a comparison and the simulated system was an aqueous solution with the addition of 0.8% by weight of citric acid and 0.08% of sodium benzoate.

Table 14 shows the values of D_{H} and PdI only in beverages after adding the self-emulsifying composition, because before the addition no nanoparticles were found that would allow measurement by the DLS method, with the exception of drink sample No. 7 (Lipton drink—tea beverage, where D_{H} and PdI were 154.7 nm and 0.319, respectively).

TABLE 14
Dispersion tests of the self-emulsifying composition in commercial beverages.

| | | | | | | Trans. | Trans. | D_{H} | |
|---|---|---|---|---|---|---|---|---|---|
| | Commercial | V | Illustrative | pH | pH | before^{a} | after^{b} | after^{b} | Pdl |
| # | name | [mL] | description | before^{a} | after^{b} | [%] | [%] | [nm] | after^{b} |
| 1 | Water | 500 | control | 6.00 | 5.67 | 100.0 | 99.2 | 37.5 ± 5.3 | 0.261 |
| 2 | Aqueous | 500 | simulated | 2.81 | 2.83 | 99.5 | 99.1 | 43.3 ± 2.3 | 0.312 |
| | solution | | system | | | | | | |
| 3 | Schweppes | 900 | tonic | 2.88 | 2.97 | 100.0 | 98.9 | 125.1 ± 89.1 | 0.361 |
| | | | drink | | | | | | |
| | | | [symbol] | | | | | | |
| 4 | Cappy | 1000 | juice | 3.82 | 3.76 | 97.2 | 96.9 | UN | 0.256 |
| | | | beverage | | | | | | |
| | | | [symbol] | | | | | | |
| 5 | Sprite | 500 | carbonated | 3.01 | 3.05 | 100.0 | 99.4 | 45.3 ± 1.4 | 0.379 |
| | | | drink | | | | | | |
| | | | [symbol] | | | | | | |
| 6 | RedBull | 250 | energy | 3.88 | 3.87 | 95.2 | 94.3 | 66.1 ± 7.0 | 0.323 |
| | | | drink | | | | | | |
| | | | [symbol] | | | | | | |
| 7 | Lipton | 330 | tea | 3.74 | 3.73 | 57.2 | 52.7 | 82.6 ± 2.8 | 0.246 |
| | | | beverage | | | | | | |
| | | | [symbol] | | | | | | |
| 8 | Heineken | 500 | beer | 4.79 | 4.85 | 97.1 | 93.2 | 163.1 ± 8.4 | 0.267 |
| | | | [symbol] | | | | | | |
| 9 | Coca-Cola | 1000 | carbonated | 3.05 | 3.06 | 37.8 | 37.8 | UN | 0.508 |
| | | | drink | | | | | | |
| | | | [symbol] | | | | | | |
| 10 | Frugo | 250 | fruit | 3.42 | 3.42 | 41.8 | 40.2 | UN | 0.988 |
| | | | drink | | | | | | |
| | | | [symbol] | | | | | | |
| 11 | OnLemon | 330 | lemonade | 3.98 | 3.94 | 24.6 | 15.5 | UN | 0.386 |
| | | | [symbol] | | | | | | |
| 12 | Cisowianka | 700 | sparkling | 7.43 | 7.67 | 98.9 | 100.0 | 46.7 ± 1.4 | 0.334 |
| | | | water | | | | | | |
| | | | [symbol] | | | | | | |

V—volume of the drink, unit packaging
^{a}the original commercial beverage before adding the self-emulsifying composition No. 80
^{b}the original commercial drink after adding the self-emulsifying composition No. 80, which provides a dose of 10 mg of CBD for the entire unit packaging
UN—no possibility of measuring/recording the results due to the physicochemical characteristics of the sample

The results show that the composition No. 80 (variant 3) provides for nanoparticle formation in commercially available beverages with a particle size within the range of D_{H}=37.5÷163.1 nm. Measurement of nanoparticle size was not possible in all cases due to the high turbidity of some beverages. In the case of alcoholic beverages, the presence of alcohol has a noticeable effect on the increase in diameter of nanoparticle in the dispersion system, which is visible in the example of Heineken beer. Nevertheless, the particle size met the acceptance criterion and was below 200 nm. In all cases of the tested beverages, no significant change in pH (above 0.5 on the pH scale) upon addition of the appropriate dose of composition 80 was observed.

Stability Tests

Storage tests were performed to determine the long-term stability of the self-emulsifying compositions and the suitability for consumption. In order to determine the longest shelf life and stability in the shortest possible test time, accelerated shelf-life tests (ASLT) were carried out. This method utilizes a kinetic model of chemical reactions that includes all factors that may affect their rate, where in fact the most common way to accelerate the reactions is to place the product at a constant, elevated temperature.

As part of the stability tests, 4 variants of the composition were analyzed (Nos. 78÷81). Each test was performed in triplicate (n=3). The stability tests over time were performed at 4° C., room temperature (21±2° C.) and at 45° C. (accelerated tests). Compositions stored for 69 days at 45° C. correspond to 12 months of storage at 21° C. After this time, the dispersion time, transmittance (T %), hydrodynamic diameter of nanoparticles (D_{H}), nanoparticle size distribution (PdI) and CBD content were determined in relation to the nominal value (initial—resulting from the sample weight).

TABLE 15
Stability of technological parameters of the self-emulsifying compositions during storage.

| | | | Sample | | | 0 days | | | | 69 days | | | |
|---|---|---|---|---|---|---|---|---|---|---|---|---|---|
| | | | | | CBD | Transmittance | D_{H} | | UHPLC | Transmittance | D_{H} | | UHPLC |
| # | Temperature | | Variant | Replicate | concentration | [%] | [nm] | Pdl | [%] | [%] | [nm] | Pdl | [%] |
| 1 | 4° | C. | 1 | 1 | 10.12% | 97.7 | 76.4 | 0.142 | 99.52 | 97.8 | 75.9 | 0.153 | 97.27 |
| 2 | | | No. 78 | 2 | 10.11% | 97.6 | 73.6 | 0.117 | 98.68 | 98.1 | 71.2 | 0.111 | 100.53 |
| 3 | | | | 3 | 9.84% | 97.3 | 75.6 | 0.121 | 100.78 | 97.6 | 74.9 | 0.123 | 99.59 |
| 4 | | | 2 | 1 | 19.93% | 92.1 | 104.5 | 0.137 | 98.75 | 90.4 | 108.4 | 0.132 | 103.66 |
| 5 | | | No. 79 | 2 | 19.97% | 89.6 | 112.3 | 0.141 | 98.47 | 91.4 | 109.1 | 0.148 | 100.63 |
| 6 | | | | 3 | 20.02% | 90.6 | 108.5 | 0.147 | 98.73 | 90.2 | 111.6 | 0.169 | 100.4 |
| 7 | | | 3 | 1 | 9.95% | 98.4 | 49.1 | 0.371 | 99.65 | 100 | 40.3 | 0.273 | 98.15 |
| 8 | | | No. 80 | 2 | 9.87% | 99.1 | 37.8 | 0.26 | 96.93 | 99.1 | 36.1 | 0.217 | 98.47 |
| 9 | | | | 3 | 10.10% | 99.7 | 35.5 | 0.213 | 99.12 | 99 | 50.1 | 0.317 | 99.54 |
| 10 | | | 4 | 1 | 20.12% | 93.5 | 115.6 | 0.193 | 100.21 | 88.7 | 131.4 | 0.168 | 98.46 |
| 11 | | | No. 81 | 2 | 19.98% | 93 | 115.1 | 0.157 | 98.97 | 90.4 | 121.8 | 0.154 | 100.18 |
| 12 | | | | 3 | 19.88% | 93.5 | 111.7 | 0.152 | 98.73 | 92.4 | 117 | 0.195 | 99.13 |
| 13 | 21 ± 2° | C. | 1 | 1 | 10.12% | 97.7 | 76.4 | 0.142 | 99.52 | 98.4 | 94.2 | 0.268 | 101.16 |
| 14 | | | No. 78 | 2 | 10.11% | 97.6 | 73.6 | 0.117 | 98.68 | 97.9 | 72.3 | 0.119 | 98.73 |
| 15 | | | | 3 | 9.84% | 97.3 | 75.6 | 0.121 | 100.78 | 98.1 | 74.3 | 0.138 | 102.02 |
| 16 | | | 2 | 1 | 19.93% | 92.1 | 104.5 | 0.137 | 98.75 | 92.8 | 106.9 | 0.125 | 97.68 |
| 17 | | | No. 79 | 2 | 19.97% | 89.6 | 112.3 | 0.141 | 98.47 | 92 | 111.1 | 0.179 | 99.02 |
| 18 | | | | 3 | 20.02% | 90.6 | 108.5 | 0.147 | 98.73 | 91.7 | 108.1 | 0.155 | 98.41 |
| 19 | | | 3 | 1 | 9.95% | 98.4 | 49.1 | 0.371 | 99.65 | 99.6 | 45.5 | 0.34 | 101.53 |
| 20 | | | No. 80 | 2 | 9.87% | 99.1 | 37.8 | 0.26 | 96.93 | 99.5 | 48.5 | 0.353 | 99.65 |
| 21 | | | | 3 | 10.10% | 99.7 | 35.5 | 0.213 | 99.12 | 99.2 | 41.5 | 0.307 | 96.66 |
| 22 | | | 4 | 1 | 20.12% | 93.5 | 115.6 | 0.193 | 100.21 | 92.5 | 143.7 | 0.229 | 97.47 |
| 23 | | | No. 81 | 2 | 19.98% | 93 | 115.1 | 0.157 | 98.97 | 93.4 | 123.2 | 0.164 | 100.25 |
| 24 | | | | 3 | 19.88% | 93.5 | 111.7 | 0.152 | 98.73 | 93.2 | 126.4 | 0.243 | 100.57 |
| 25 | 45° | C. | 1 | 1 | 10.12% | 97.7 | 76.4 | 0.142 | 99.52 | 96.3 | 73.7 | 0.134 | 98.03 |
| 26 | | | No. 78 | 2 | 10.11% | 97.6 | 73.6 | 0.117 | 98.68 | 98.5 | 70.1 | 0.135 | 98.33 |
| 27 | | | | 3 | 9.84% | 97.3 | 75.6 | 0.121 | 100.78 | 97.9 | 71.7 | 0.125 | 100.64 |
| 28 | | | 2 | 1 | 19.93% | 92.1 | 104.5 | 0.137 | 98.75 | 94.2 | 100.8 | 0.137 | 99.46 |
| 29 | | | No. 79 | 2 | 19.97% | 89.6 | 112.3 | 0.141 | 98.47 | 91.1 | 109.7 | 0.131 | 99.1 |
| 30 | | | | 3 | 20.02% | 90.6 | 108.5 | 0.147 | 98.73 | 94.3 | 101.4 | 0.113 | 99.34 |
| 31 | | | 3 | 1 | 9.95% | 98.4 | 49.1 | 0.371 | 99.65 | 99.4 | 35.8 | 0.36 | 96.06 |
| 32 | | | No. 80 | 2 | 9.87% | 99.1 | 37.8 | 0.26 | 96.93 | 99.4 | 34.7 | 0.214 | 96.32 |
| 33 | | | | 3 | 10.10% | 99.7 | 35.5 | 0.213 | 99.12 | 99.7 | 37.2 | 0.282 | 97.3 |
| 34 | | | 4 | 1 | 20.12% | 93.5 | 115.6 | 0.193 | 100.21 | 92 | 118.7 | 0.178 | 99.43 |
| 35 | | | No. 81 | 2 | 19.98% | 93 | 115.1 | 0.157 | 98.97 | 93.1 | 117.2 | 0.185 | 98.99 |
| 36 | | | | 3 | 19.88% | 93.5 | 111.7 | 0.152 | 98.73 | 93.8 | 112.9 | 0.171 | 93.7 |

Based on Table 15 regarding the stability of self-emulsifying compositions over the studied storage time, along with the accelerated shelf-life tests, the minimum shelf life of the product that maintains the physicochemical, technological and process parameters of the compositions disclosed is 12 months (as confirmed in the studies), where in order to maintain a dose of cannabinoids or relevant cannabinoid extract, it is recommended to use an excess of 5% as the active substance.

Procedure for the Preparation of Nanoemulsions at the Industrial Scale

The production of nanoemulsion at the industrial (semi-technical) scale was carried out on a Kates thermostatic mixer with a volume of 5 liters, equipped with a single blade impeller powered by a 0.75 kW Basel S.A Cantoni® Group motor controlled by an inverter.

The nanoemulsions were prepared by pouring 3500 mL of water into the mixer, to which 1400 mg of the composition containing: T80:MCT:MCM:CBD, 50:20:20:10% by weight was added. The mixture was mixed for 1 min at 17% of engine power for the variant 1 and at 55% of engine power for the variant 2. After this time, the samples were analyzed. In both cases, the temperature during the process was 23.1±0.2° C. as indicated by the temperature sensor of the mixer.

TABLE 16
Study może lepiej Evaluation of nanoemulsions
obtained at the industrial scale.

| | | Mixing | Transmittance | | |
|---|---|---|---|---|---|
| Nr | Name | power | [%] | D_{H} [nm] | Pdl |
| 78 | Variant 1 | 17% | 99.8 ± 0.1 | 34.1 ± 0.2 | 0.175 ± 0.007 |
| 79 | Variant 2 | 55% | 99.9 ± 0.1 | 32.4 ± 0.2 | 0.156 ± 0.024 |

The preferred examples of the described compositions are scalable and form nanoemulsions at both laboratory and industrial scale. The results summarized in Table 16 show that the transparency of the solutions measured by transmittance (% T), the nanoparticle hydrodynamic diameter and the polydispersity of the systems obtained at a semi-technical scale meet the above-mentioned requirements. In both cases (78 and 79) the transmittance is close to 100% and the hydrodynamic diameter does not exceed 35 nm. Both systems are also monodisperse, and the PdI value does not exceed 0.180.

The type of mixing used and the mixing speed have an impact on the quality of the nanoemulsion, but the advantage of the claimed compositions is that desired nanoemulsion parameters can be obtained with minimal mixing.

The studied compositions containing Tween™ 80 as a surfactant in the amount of 50% by weight also confirmed that the surfactant content did not adversely affect the nanoemulsion formation process itself and did not cause foaming. Foaming is an undesirable effect and is a common technological problem, especially at the industrial scale. The described compositions and method of use thereof do not have this problem even at the highest surfactant concentrations.

1. A composition containing a cannabinoid or cannabinoid extract, a surfactant and two different lipid fractions.

2. The composition of claim 1, characterized in that the cannabinoid or cannabinoid extract constitutes no more than 20% by weight of the composition, the surfactant constitutes 30-50% by weight of the composition, the first lipid fraction constitutes 20-35% by weight of the composition, the second lipid fraction constitutes 15-30% by weight of the composition.

3. The composition of claim 1, characterized in that the cannabinoid is a compound selected from the group consisting of: Δ⁹-tetrahydrocannabinol (Δ⁹-THC), cannabidiol (CBD), tetrahydrocannabinolic acid (THC-A), cannabidiolic acid (CBDA), cannabinol (CBN), cannabigerol (CBG), cannabigerolic acid (CBG-A), cannabichromene (CBC), tetrahydrocannabivarin (THC-V), Δ⁸-tetrahydrocannabinol (Δ⁸-THC), cannabidivarin (CBDV) or cannabicycol (CBL), or mixtures thereof.

4. The composition of claim 1, characterized in that the cannabinoid extract is an extract of Cannabis sativa, Cannabis indica, Cannabis hybrid or another species of the genus Cannabis or mixtures thereof.

5. The composition of claim 1, characterized in that the surfactant is polysorbate 80, the first lipid fraction is a C8÷C12 medium chain length triacylglyceride and the second lipid fraction is a C8÷C12 medium chain mono-diacylglyceride.

6. The composition of claim 1, characterized in that it further comprises a substance selected from the group consisting of: water and preserving, sweetening, colouring or pH-stabilizing additives suitable for use in pharmaceutical or food products.

7. Use of the composition of claim 1 for preparation of a water-based oral product, preferably a pharmaceutical composition, a medical product or a food product.

8. A method for production of a stable monodisperse system, characterized in that water or an aqueous solution is mixed with the composition as defined in claim 1.

9. The method claim 8, characterized in that the mixing time is less than 120 seconds, preferably less than 60 seconds, with mixing intensity not exceeding 2500 RPM, preferably with mixing intensity not exceeding 150 RPM.

10. A monodisperse system, characterized in that it consists of a dispersion medium being water or an aqueous solution and a dispersed phase formed by the particles obtained from the composition as defined in claim 1, wherein the particles preferably have an outer layer containing a surfactant, and an inner layer which is a mixture of lipids in which a cannabinoid or cannabinoid extract is dissolved.

11. The monodisperse system of claim 10, characterized in that the dispersed phase is formed by particles smaller than 180 nm.

12. The monodisperse system of claim 10, characterized in that it has a transparency of more than 85%, preferably more than 90%.

13. The monodisperse system of claim 10, characterized in that it has a monodispersity below 0.250.

14. The monodisperse system of claim 10, characterized in that it maintains the nanoemulsion stability upon dilution up to 10,000× with water or an aqueous solution.

15. The monodisperse system of claim 10, characterized in that it maintains the nanoemulsion stability over the pH range from 8 to 3.
